(19) United States
(12) Patent Application Publication
(10) Pub. No.: US 2026/0102273 A1
(43) Pub. Date: Apr. 16, 2026

(54) SPLINTS AND METHODS OF TREATMENT

(71) Applicant: PS Medical Services Limited, Kumeu (NZ)

(72) Inventor: Pranesh Kumar, Kumeu (NZ)

(21) Appl. No.: 19/356,246

(22) Filed: Oct. 13, 2025

(30) Foreign Application Priority Data
Oct. 11, 2024 (AU) 2024903286

(51) Int. Cl. A61F 5/058

(52) U.S. Cl. CPC A61F 5/05866

(57) ABSTRACT

A splint is provided for treating a patient's joint having an axis of rotation, such as the carpometacarpal (CMC) joint of the thumb. The splint includes a cradle and an anchor positioned respectively distal and proximal to the joint's axis of rotation. In use, the cradle and anchor bear against corresponding body surfaces to position the bones forming the treated joint in a desired orientation and at least partially restrict undesired movement. The cradle and anchor together define a lever arm that produces a traction force on muscle contraction, aiding in joint alignment and stabilization. The splint may further include a fastener, liner, flexible bridge, and sling region to improve comfort, fit, and controlled flexibility.

### CROSS-REFERENCE TO RELATED APPLICATION

This application claims the benefit of and priority to Australia (AU) Patent Application No. 2024903286 filed Oct. 11, 2024, the contents of which being incorporated by reference in their entirety herein.

### TECHNICAL FIELD

The present disclosure relates to splints and methods of treatment.

### BACKGROUND

Many medical conditions require joints to be placed into a desired position to alleviate symptoms and/or promote healing.

Many of these medical conditions are caused by or acerbated by the natural tension applied by tendon(s) through the joint. For instance, shortening of a tendon can cause a diseased joint or fractured bone to be moved out of alignment. Essentially, the tension applied by the tendon may prevent fracture reduction or move a joint into an unwanted position which undermines optimal healing and/or creates unwanted pain and discomfort.

Furthermore, it may be beneficial to place a joint in a certain position to facilitate normal function of a person's limb(s) for instance while alleviating symptoms, improving function of the immediate or surrounding joints and/or promoting healing. For instance, treating a hallux valgus deformity is is a common medical conditions for which it is useful to place a joint in a desired orientation.

Arthritis is a common medical condition, which is increasing in prominence.

Several types of arthritis are known, with the two most common being osteoarthritis and rheumatoid arthritis. Osteoarthritis is a degenerative disease that generally gets worse over time. It causes the cartilage of the joint(s), which normally facilitate movement of the joint articulating surfaces relative to each other, to degrade.

In addition, people suffering from osteoarthritis often suffer from shortening tendons. These apply pressure onto the bones of the affected joint(s). As the disease progresses the tendons shorten and cause the joint to deform, which causes patient discomfort and pain.

There is no known cure for osteoarthritis.

Each type of arthritis can affect any joint in the body. One of these is the basal joint of the thumb, which is known as the carpometacarpal (CMC) joint. FIG. 1 shows the location of the CMC joint, which can be generally described as being located under the fleshy part of the thumb near the wrist.

The CMC joint is critical to normal function of the hand. It allows the thumb to swivel, pivot and pinch (grab). Therefore, any condition which affects normal function of the thumb or which causes pain through use of the thumb has a significant affect on the quality of a patient's life.

Common symptoms of arthritis of the CMC joint include:
Pain with activities that involve gripping or pinching, such as turning a key, opening a door, or snapping your fingers.
Loss of strength in gripping or pinching activities.
Swelling and tenderness at the base of the thumb.
Aching discomfort after prolonged use of the thumb.
An enlarged, “out-of-joint” appearance at the base of the thumb.
Development of a bony prominence or bump over the basal joint at the base of the thumb.
Limited or restricted motion in the thumb.

Arthritis is commonly treated by splinting the joint. This involves fitting a splint to the limb, which holds the joint in, or towards, a desired position. These splints may also restrict movement of the joint. A splint used to treat an arthritic CMC joint may hold the thumb in or towards abduction.

However, available splints are unsatisfactory, for one or more of the following reasons:
They are bulky and cumbersome. This can affect patient compliance (which in turn reduces treatment efficacy) or hinder the patient's normal life and participation in activities.
Being uncomfortable to wear. Again, this can affect patient compliance and treatment efficacy.
Reducing the range of motion in the joint in an undesirable way. That can limit a patient's ability to perform tasks or activities, either affecting quality of life or reducing compliance.

Frequently surgery is used to treat arthritis where the non-surgical options are ineffective. One surgical approach is to fuse the bones of the joint. That limits movement of the joint across the damaged joint surfaces - and therefore reduces pain. However, it also reduces the range of movement which can be undesirable.

Another surgical approach is to repair the joint, using cartilage and tendon from healthy joints. However, this approach may not be viable for all patients.

Irrespective of the benefits surgical treatment may provide, they generally require a general anaesthetic. That may make them unsuitable for many patients. In addition, they have extended recovery times, can be relatively costly or they may not be readily available e.g. due to limited, or competing demand for, surgical resources.

It would therefore be beneficial to have an improved splint which addresses one or more of the foregoing problems.

Alternatively, or in addition, it is an object of the present technology to provide the public with a useful choice.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### BRIEF SUMMARY OF THE TECHNOLOGY

The present specification describes improvements to splints and methods of treating medical conditions using the splints. The technology may find application with patients, and optionally humans and non-human animals.

Representative conditions that may be treated include arthritis (osteoarthritis or rheumatoid arthritis), limb and joint deformities such as a hallux valgus deformity, or fractures such as a fracture of the fifth metacarpal bone of the hand near the knuckle (commonly referred to as a boxer fracture).

Features of the technology will be described with reference to representative embodiments. However, it should be appreciated that these are non-limiting on the scope of the technology.

According to an aspect of the technology, there is provided a splint to treat a patient's joint, wherein the joint has an axis of rotation, wherein
the splint includes a cradle and an anchor, and wherein
the splint is structured and/or arranged to, in use, be positioned with respect to the joint so that:
the cradle is adjacent to and bears against a surface of the patient's body that is distal to the axis of rotation, and
the anchor is adjacent to and bears against a portion of the patient's body that is proximal to the axis of rotation; and further wherein
the cradle and the anchor together define a lever arm which provides a traction force to the joint on contraction of muscles around the joint.

According to another aspect of the technology, there is provided a splint to treat a patient's joint, wherein
the splint includes a cradle and an anchor, and wherein
the splint is structured and/or arranged to, in use, be positioned with respect to the joint so that:
the cradle is adjacent to and bears against a surface of the patient's body that is distal to the joint, and
the anchor is adjacent to and bears against a portion of the patient's body that is proximal to the joint; and further wherein
the splint is configured to position bones forming the joint in a desired orientation and at least partially restrict movement at the joint away from the desired orientation.

According to another aspect of the technology, there is provided a splint for a carpometacarpal (CMC) joint of a patient, wherein the splint includes
a cradle that is configured to be positioned adjacent to and bear against a surface of the patient's thumb which is distal to the CMC joint, and wherein
the splint has an anchor which is configured to be positioned adjacent to and bear against a surface of the patient's body which is on the proximal side of the CMC joint, and wherein
the splint is configured to hold the patient's thumb in a desired position and at least partially restrict movement of the thumb at the CMC joint in a direction that is into or towards one or more of flexion and adduction.

According to another aspect of the technology, there is provided a splint for a boxer's fracture, wherein the splint includes
a bearing portion configured to apply pressure to one or more bone fragments of the boxer's fracture to reduce the fracture, and
a cradle that is configured to be placed adjacent to and bear against a surface of the patient's body that is distal to the fifth metacarpal-phalangeal joint, and
the anchor is configured to be placed adjacent to and bear against a portion of the patient's body that is proximal to the fracture; and further wherein
the cradle and the anchor together create a lever arm which provides a traction force to the joint on contraction of the muscles around the joint.

According to another aspect of the technology, there is provided a splint for a hallux valgus deformity, wherein the splint includes
a cradle that is configured to be positioned against a surface of the the patient's hallux which is distal to the metatarsal-phalangeal joint of the hallux, and wherein
the splint has an anchor which is configured to bear against a surface of the patient's body which is proximal to the metatarsal-phalangeal joint, and wherein
the splint is configured to hold the patient's metatarsal-phalangeal joint in a desired position and at least partially restrict movement of the metatarsal-phalangeal joint in a direction that is into or towards hallux valgus.

Throughout the present specification reference will be made to a “patient”. This should be understood as meaning an animal with which the splint is to be used e.g. a human or a non-human animal. Reference to the patient should not be seen as limiting as features of the present technology may find application with one or more class of patient.

Throughout the present specification, reference will be made to a “joint”. This should be understood as meaning a junction between two adjacent structural elements in the skeleton of an animal. In normal physiology, the joints with which the present technology facilitate movement between the adjacent structural element.

In some forms, the joint may be a synovial joint, and optionally a synovial joint such as a saddle joint, a ball and socket joint, a condyloid joint, or a hinge joint. Therefore, it should be understood that the present technology is not limited to only one type of joint.

Throughout the present specification reference will be made to the term “treated joint”. This should be understood as meaning a joint of the patient which requires treatment due to a medical or other condition.

Throughout the present specification reference will be made to the term “adjacent joint”. This should be understood as meaning joint of the patient that is adjacent to the treated joint.

Examples of the treated joint and adjacent will be discussed with reference to specific embodiments of the present technology.

In some embodiments, the splint may be used to treat a joint which is part of a joint series that comprises a first joint and a second joint e.g. the treated joint is the first joint which is proximal and the adjacent joint is the second joint and it is distal. For instance, in an embodiment where the splint is used to treat the CMC joint (as described herein) the first joint may be the CMC joint (and it is therefore the treated joint) and the second joint may be the first metacarpal-phalangeal joint (and it is therefore the adjacent joint).

In yet a further example, in an embodiment where the splint may be used to treat a boxer fracture, the first joint may be the metacarpo-phalangeal joint and the second joint may be the inter-phalangeal joint.

These aspects of the present technology should become clearer from the following description. However, it should be understood that the description herein and the embodiments are not limiting on the scope of the present technology.

In an embodiment, the technology may be used to treat a fracture of a bone forming part of a joint, and reference herein will be made to this embodiment as a splint for a fracture. For instance, a splint for a fracture may be used to treat a boxer fracture or other fracture in which it is beneficial to obtain the benefits of the present technology.

In addition, or the alternative, the present technology may be used to treat a patient that does not have a fracture in a bone in or near the treated joint. For instance, the technology may be used to treat an arthritic joint e.g. the CMC joint or a deformed joint e.g. a hallux valgus deformity.

It should be understood that the foregoing description should not be seen as limiting as the present technology can be used to treat different types of medical conditions as should be appreciated by one skilled in the art.

Throughout the present specification reference to the term “cradle” should be understood as meaning a portion of the splint which can bear against a surface of the patient's body which is distal to the joint.

In some embodiments, the cradle is the portion of the splint which is in contact with, and therefore applies pressure to, a surface of the patient's body to position the joint in a desired orientation.

It is envisaged that the present technology may have one distinct cradle or a plurality of cradles which together position the joint in the desired orientation. Therefore, the foregoing discussion should not be seen as limiting on the scope of the present technology.

In an embodiment, the cradle may have a distal edge.

Throughout the present specification, reference to the term “distal edge” should be understood as meaning a feature against which a patient's body can press. In use, the action of the patient's muscles that cause movement e.g. flexion in the joint causes the body to press against the distal edge.

The distal edge may assist in creating a traction force as is discussed in more detail below.

Throughout the present specification reference to the term “anchor” should be understood as meaning a component that can bear against a surface of a patient's body to provide a point of fixation for the splint.

In some embodiments, the anchor may be configured to bear against a surface of the patient's body that is proximal to the treated joint. As a result, the splint may be better able to position the joint in a desired orientation and/or create a traction force that can be applied to the joint.

In some embodiments, the anchor may be formed from or structured to be, relatively rigid. This may facilitate positioning the joint in a desired orientation and/or creating a traction force that is applied to the joint.

Throughout the present specification reference to the term “axis of rotation” should be understood as meaning an imaginary axis that defines the native range of movement for a joint e.g. it is the axis about which the bones of a joint move in a healthy joint which is not treated with a splint according to the present technology.

This aspect of the present technology should become clearer from the following description.

The axis of rotation is different for each joint with which the present technology may be used. Once skilled in the art would understand the orientation and nature of movement with respect to that axis, for each potential joint.

Throughout the present specification reference to the term “traction force” should be understood as meaning a force which is applied to a treated joint.

The traction force may optionally be created by the splint restricting or preventing movement of at least two bones forming the treated joint.

In some embodiments, a splint according to the present technology may create and apply a traction force to the treated joint using contraction of the muscles that cause movement of the adjacent joint.

Throughout the present specification reference to the term “second anchor” should be understood as meaning a component which can be positioned adjacent to and bear against a surface of the patient's body which is distal to the joint and spaced apart from the surface against which the cradle bears.

The second anchor may be beneficial to assist in limiting movement of the treated joint. For instance, it may act as a stop to limit or prevent movement of the cradle beyond a point on contraction of muscles to cause movement at the joint.

Throughout the present specification reference to the term “bridge” should be understood as meaning a structure to which the cradle is attached, connected or formed, and which provides separation between the cradle and the body portion.

The provision of a bridge may assist in creating a splint which has a desired range of motion, to improve the positioning of the cradle in use, to reducing the overall size or bulk of the splint.

However, it is also envisaged that the splint may not have a distinct bridge structure.

Throughout the present specification, reference will be made to “restrict movement”. This should be understood as meaning to limit or provide resistance to movement in some way e.g. in at least one direction or plane of movement. Restricting movement should be understood as having a similar meaning.

In some embodiments, the present technology may allow some movement in the direction or plane of movement in which movement is restricted. For instance, the technology may be structured and/or arranged to allow force applied to a bone forming part of a joint to at least partially overcome the restriction provided by the splint. This may be achieved by features such as the resiliency of the splints described herein or other features of the splint.

The restriction on movement should become clearer from the following description.

Throughout the present specification, reference will be made to a “desired position”. This should be understood as meaning an orientation for a bone of the treated joint against which the cradle applies relative to the other bone(s) of the treated joint.

The desired position may be any position for the bone e.g. the bone is positioned so that the joint is a neutral position, or in or towards extension, flexion, adduction or abduction. One skilled in the art would understand that the desired position is dependent on the nature of the treated joint and medical condition that is being treated. Accordingly, any description herein should not be seen as limiting.

In embodiments, the desired position is towards, or completely in, one or more of extension and abduction.

In embodiments, the desired position is at least partially in extension.

In embodiments, the desired position is at least partially in abduction.

In embodiments, the bridge may connect the cradle to one or more of a/the first arm, a/the second arm and a/the second anchor.

In embodiments, at least one of the cradle and the bridge has a degree of resiliency.

The bridge and/or cradle may provide a number of advantages. For instance, at least one of the structure, shape or resiliency (flex) of the bridge and/or cradle may assist with fitting the splint to a range of patients. In addition or the alternative, the bridge and/or cradle may allow for desired movement range for the bone(s) of the treated joint with the splint fitted e.g. towards abduction of the CMC joint on to flexing of the first metacarpal-phalangeal joint of the thumb.

In embodiments, the degree of resiliency of the cradle and the bridge is relatively minimal.

In embodiments, the cradle may include a sling.

In embodiments, the sling may be a region of the cradle which has a relatively higher degree of flexibility compared to other regions of the cradle.

In embodiments, the sling may be provided by layer of material attached to a region of the body portion that defines the cradle.

In embodiments, the layer of material extends beyond an edge of the cradle and is unsupported on an inner surface and an outer surface and along a side edge of the sling.

In embodiments, the splint may include a body portion.

In embodiments, the body portion may be shaped to define an aperture which is configured to receive a portion of the patient's body. For instance, the aperture may be configured to receive a patient's thumb and facilitate the cradle, anchor and second anchor being positioned against surfaces of the patient's hand and thumb.

In embodiments, the body portion may provide one or more of the anchor, a/the first arm, a/the second arm, and a/the second anchor of the splint.

In embodiments, the body portion may be formed as an integral, one-piece component.

In embodiments, the body portion may be formed from two or more components that are connected together.

In embodiment, the splint may further comprise a fastener configured to facilitate attachment of the splint to the patients'body and holding the splint in position with respect to the treated joint.

In embodiments, the fastener may include a strap.

In embodiments, the strap may connect to a slot formed in a/the body portion of the splint.

In embodiments, the strap may comprise a length adjustment mechanism.

In embodiments, the splint may include a liner.

In embodiments, the liner may be formed from a material which is at least one of soft, flexible, water resistant, and breathable.

Further features of the present technology should become clearer from the following non-limiting description of specific embodiments.

### CMC Splint

Throughout the present specification reference will be made to a CMC Joint Splint. This should be understood as meaning a splint for use with a patient's CMC joint.

In a CMC Splint according to the technology, the cradle may be configured to bear against a surface of the patient's thumb that is distal to the CMC joint e.g. the first web space of the patient's hand.

In a CMC Splint according to the technology, the cradle may be configured to bear against a surface of the patient's thumb that is between a proximal phalanx and a first metacarpal which form part of the thumb with which the splint is used.

In a CMC Splint according to the technology the cradle may at least partially wraps around the circumference of the patient's thumb.

In a CMC Splint according to the technology, the cradle may be configured to at least partially wrap around the proximal phalanx of the patient's thumb.

In a CMC Splint according to the technology, the cradle may be defined by a first wing and a second wing, and Withe first wing and the second wing are configured to at least partially wrap around the patient's thumb.

In a CMC Splint according to the technology, the first wing and the second wing wrap may around a respective portion of the patient's thumb and connect on the proximal side of the proximal phalanx of the thumb.

In a CMC Splint according to the technology, the cradle may be structured to allow movement of the thumb from the desired position further towards or into one or more of extension and abduction.

In a CMC Splint according to the technology, the splint may be shaped and/or arranged to allow articulation at the first metacarpo-phalangeal joint of the patient's thumb. For instance, the cradle may not substantially restrict or prevent movement at the first metacarpo-phalangeal joint.

In a CMC Splint according to the technology, the splint may be configured to, on flexion at the first metacarpal-phalangeal joint apply a traction force to the proximal phalange of the thumb which lifts the metacarpal of the thumb in the CMC joint.

In a CMC Splint according to the technology, the splint may be configured to allow movement of the patient's thumb from the desired position further towards, or into, one or more of extension and abduction.

In a CMC Splint according to the technology, the splint may include at least one arm which extends away from the anchor and is configured to conform to a surface of the patient's body e.g. the hand.

In a CMC Splint according to the technology, the splint may include at least a first arm and a second arm, and wherein the first arm and the second arm are each configured to conform to a respective one of the volar surface and the dorsal surface of the patient's hand in the area around the patient's CMC joint.

In a CMC Splint according to the technology, the first arm maybe shaped and/or configured to conform to an inner surface of the patient's body e.g. the hand in a region adjacent the CMC joint.

In a CMC Splint according to the technology, the first arm may have a curved shape which allows it to confirm to a surface of the patient's body e.g. the volar surface of the patient's hand in the region adjacent to the CMC joint.

In a CMC Splint according to the technology, the second arm may be shaped and/or configured to conform to a dorsal surface of the patient's hand in a region adjacent the CMC joint.

In a CMC Splint according to the technology, the second arm may have a curved shape which allows it to conform to the outer surface of the patient's hand in a region adjacent the CMC joint.

In a CMC Splint according to the technology, the splint may include a second anchor which is configured to, in use, bear against a surface of the patient's hand which is distal to a joint between the proximal phalanx of the patient's index finger and the 2^{nd} Metacarpal bone of the patient's hand.

Additional features of the present technology should be apparent from drawings, detailed description and claims which provide non-limiting embodiments.

### BRIEF DESCRIPTION OF THE FIGURES

Specific embodiments of the invention will now be described by way of example only, and with reference to the accompanying drawings in which:

FIG. 1 is a front view of a patient's hand indicating the structure and location of the carpometacarpal (CMC) joint;

FIG. 2A is a front cross-sectional view of a patient's hand indicating directions of movement of the CMC joint;

FIG. 2B is a rear cross-sectional view of a patient's hand with the bones of the hand labelled;

FIG. 3A is a lateral side view of a patient's hand with a CMC joint in abduction;

FIG. 3B is a front view of a patient's hand with a CMC joint in adduction;

FIG. 3C is a oblique view of a patient's hand with a CMC joint in extension;

FIG. 4 is a first view of a body portion of a splint according to a first aspect of the present technology;

FIG. 5 is a second view of the body portion of a splint of FIG. 2;

FIG. 6 is a third view of the body portion of a splint of FIG. 2;

FIG. 7 is a fourth view of the body portion of a splint of FIG. 2;

FIG. 8 is a first exploded view of a splint according to the present technology;

FIG. 9 is a second exploded view of a splint according to the present technology;

FIG. 10 is a third exploded view of a splint according to the present technology;

FIG. 11 is a fourth exploded view of a splint according to the present technology;

FIG. 12 is a first view of a splint according to the present technology;

FIG. 13 is a second view of a splint according to the present technology;

FIG. 14 is a third view of a splint according to the present technology;

FIG. 15 is a fourth view of a splint according to the present technology;

FIG. 16A is a medial side view of a splint according to the present technology fitted to a patient's hand with a CMC joint in extension;

FIG. 16B is a front view of a splint according to the present technology fitted to a patient's hand with a CMC joint in extension;

FIG. 16C is a front view of a splint according to the present technology fitted to a patient's hand with a CMC joint in abduction;

FIG. 17 is a front view of a split according to the present technology fitted to a patient's hand;

FIG. 18 is a plan view of a split according to the present technology fitted to a patient's foot;

FIG. 19A is a plan view of a patient's foot in a normal (undeformed) state;

FIG. 19B is a plan view of a patient's foot with a mild hallux valgus deformity; and

FIG. 19C is a plan view of a patient's foot with a relatively severe hallux valgus deformity.

### DETAILED DESCRIPTION

### Anatomy of a Patient's Hand and Thumb

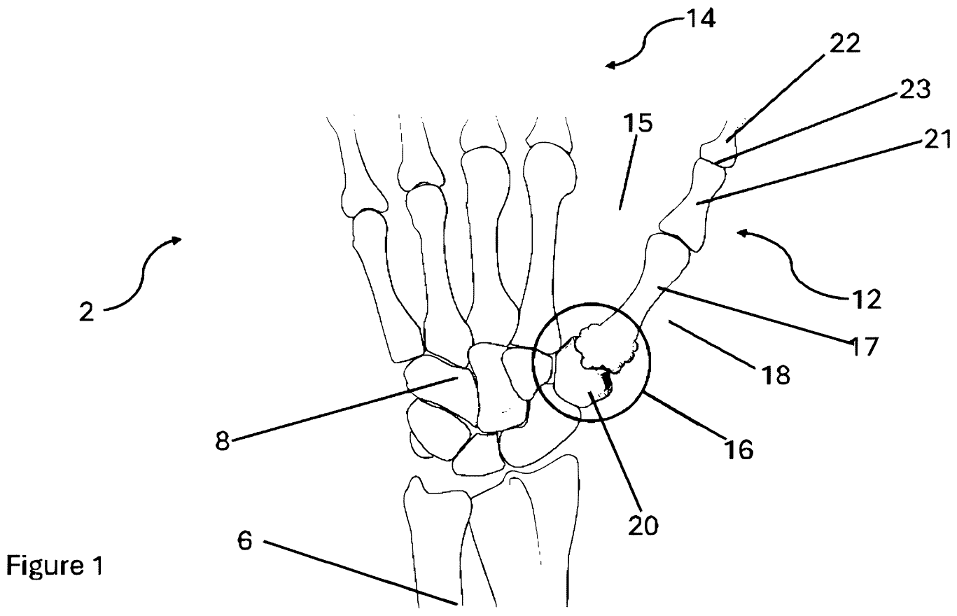

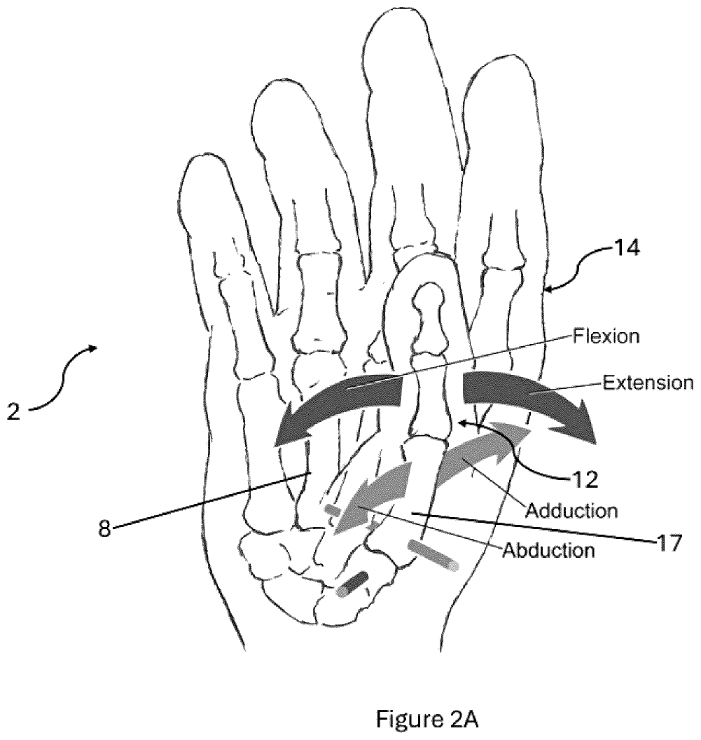

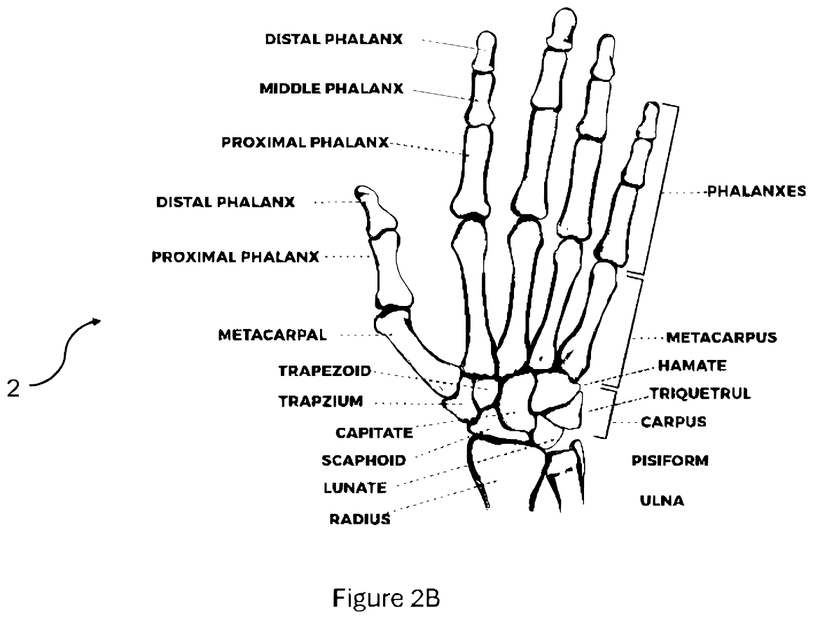

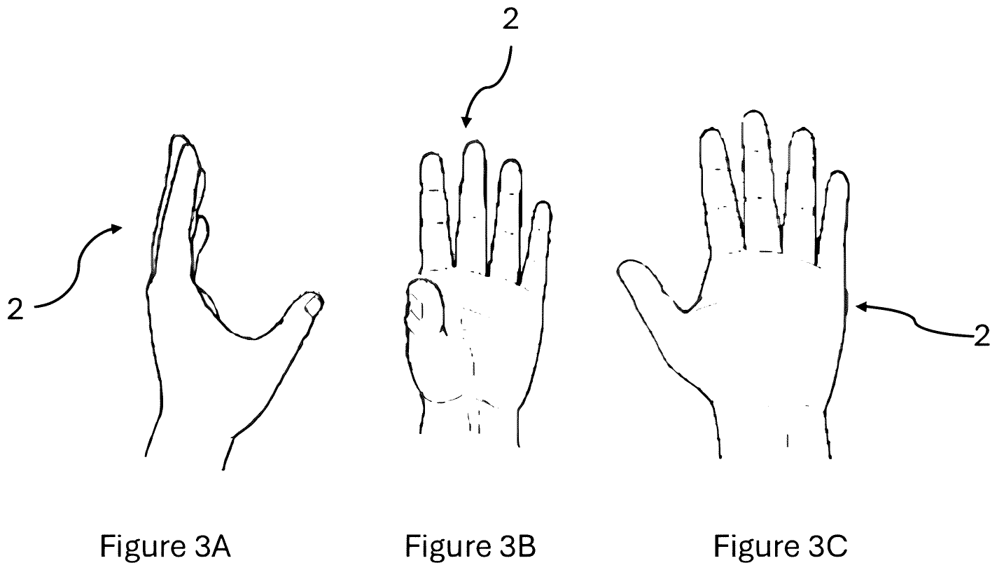

Referring first to FIGS. 1, 2A and 2B and 3A-3C, which provide reference and context for an embodiment of the present technology.

A patient's hand 2 is the limb on the distal side of the wrist 4 from the arm 6. It includes a palm 8 e.g. the inner surface of the hand 2, and a rear surface (unnumbered) e.g. the opposite side of the hand 2 to the palm 8, a thumb 12, and an index finger 14.

The thumb 12 is formed from a metacarpal 17, a proximal phalanx 21, and a distal phalanx 22.

The thumb 12 has an inner surface 15 being the general area between the first metacarpal 17 and the index finger 14 when the thumb 12 lies against the index finger 14 e.g. is in extension and adduction. The thumb 12 also has an outer surface 18 being the surface of the thumb on the distal side of the first metacarpal 17 from the inner surface 15.

The thumb 12 is connected to the hand 2 at the carpometacarpal (CMC) joint, indicated generally as 16. The CMC joint 16 is the defined by the articulating surfaces of the trapezium 20 and the first metacarpal 17.

The thumb also includes a joint 23 between the proximal phalanx 21 and the distal phalanx 22, which is known as the inter-phalangeal joint and will be referred to as such.

It should be understood that the foregoing discussion is not exhaustive of the bones and structure of the hand 2. Instead, it is provided only to facilitate additional understanding of the present technology and its benefits, and should therefore not be seen as limiting on the scope of the present technology.

### First Embodiment of a Splint

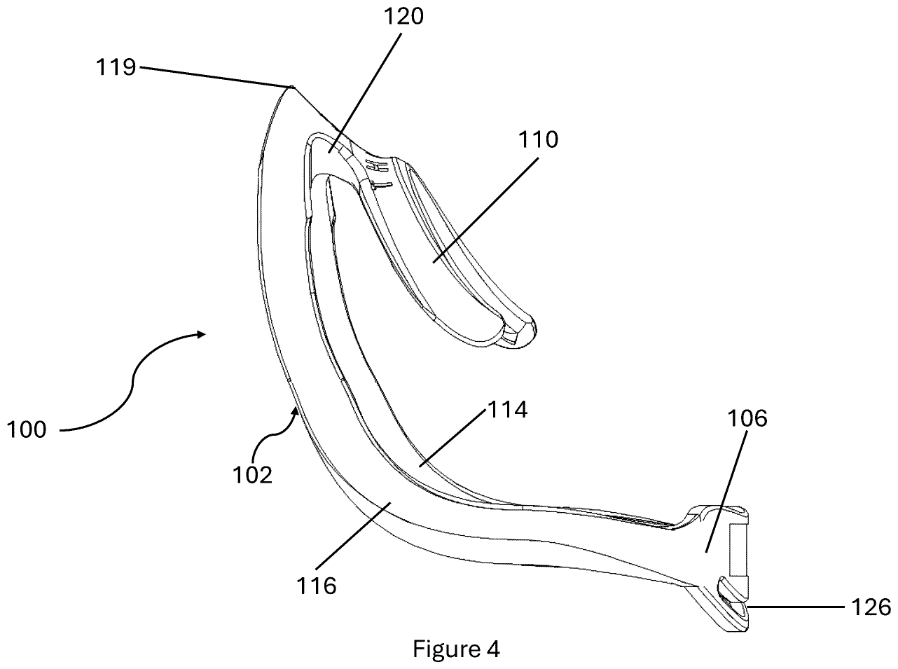

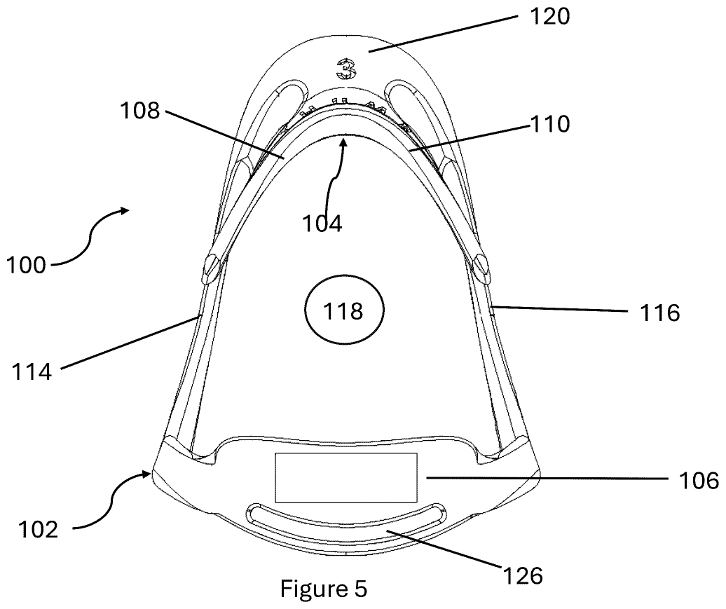

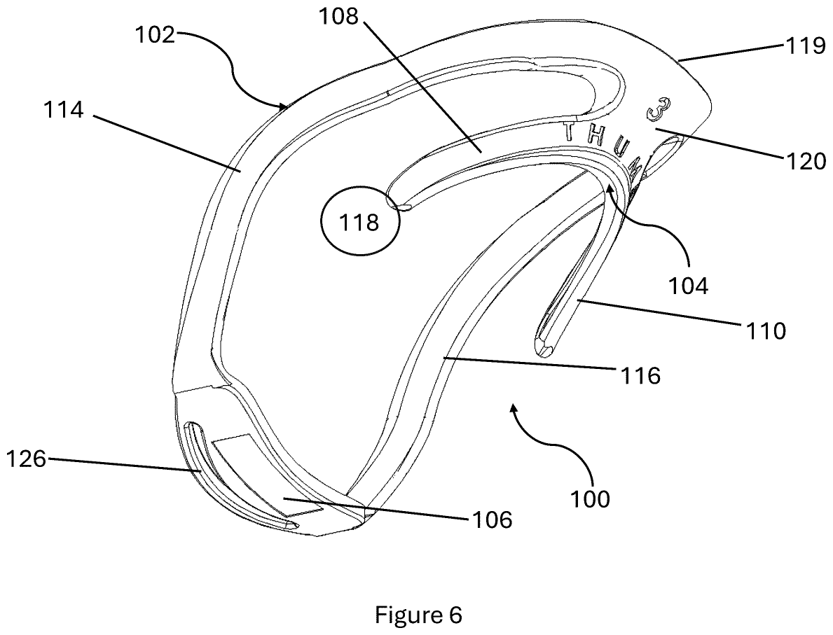

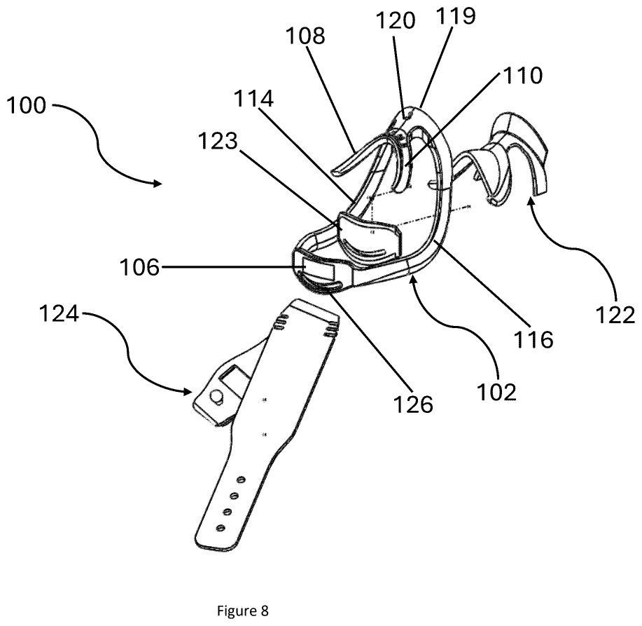

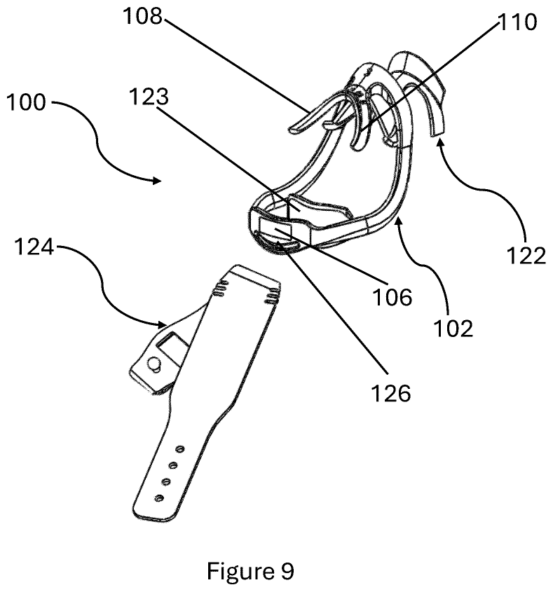

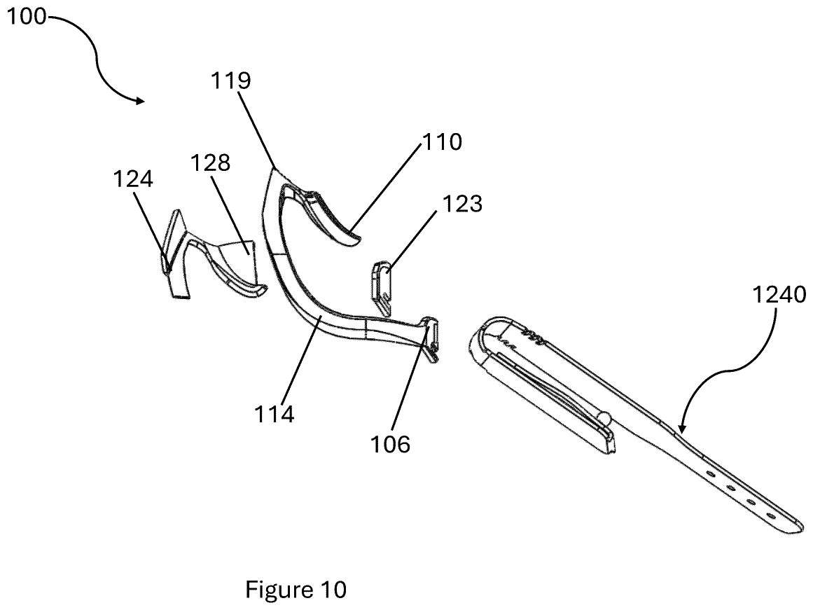

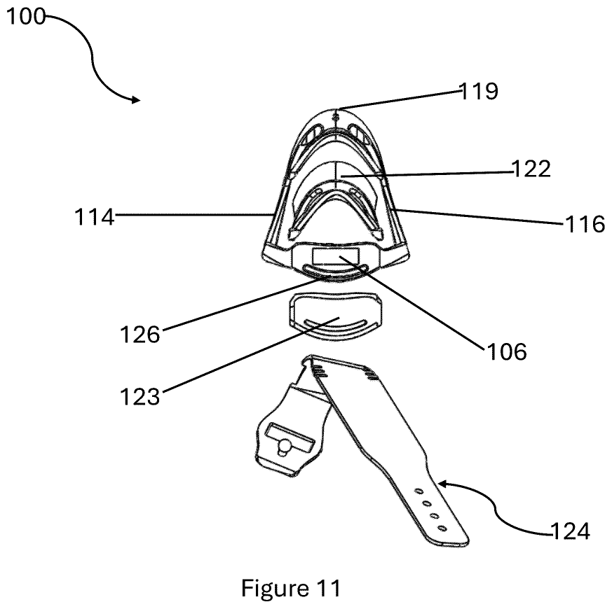

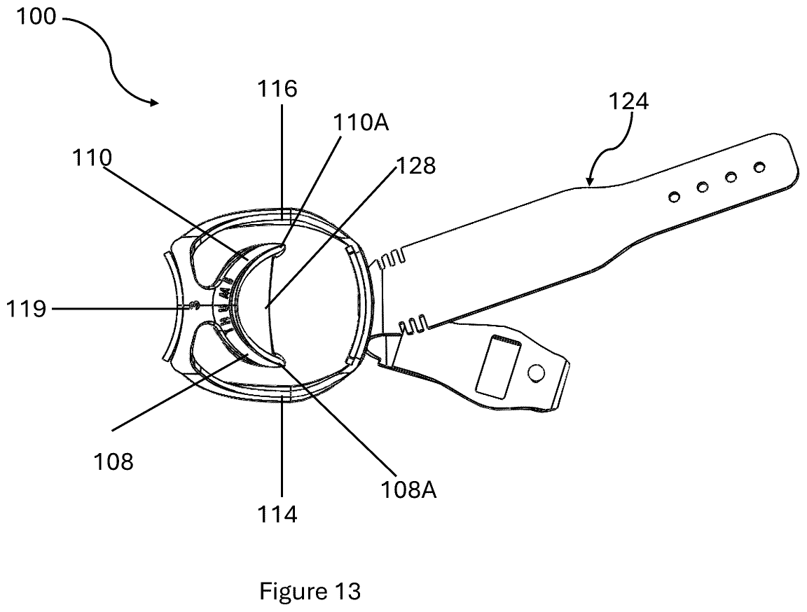

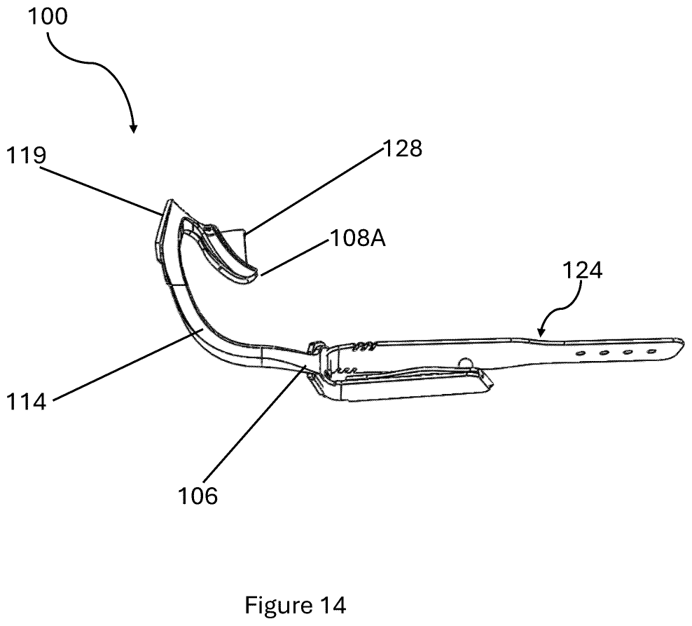

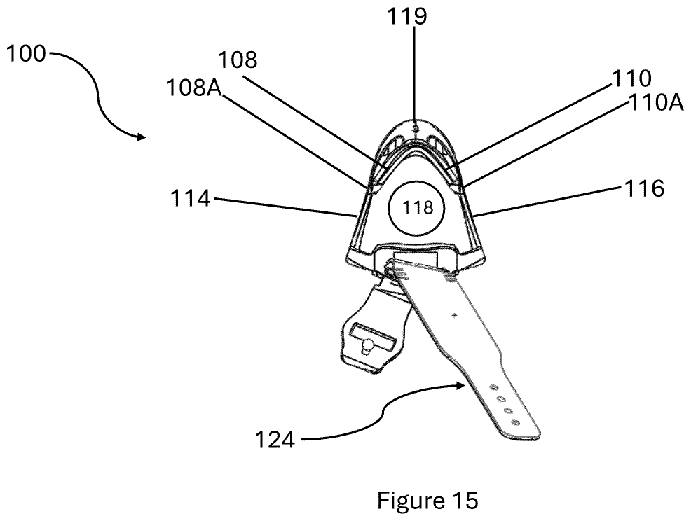

Referring now to FIGS. 4 to 15 which show views of a splint according to an embodiment of the present technology, in the form of a splint 100 that is configured for treatment of arthritis of the carpometacarpal (CMC) joint 16 e.g. osteoarthritis. For instance, the splint 100 may reduce or eliminate symptoms experienced by a patient with arthritis in the CMC joint 16. In addition, the splint 100 may improve the functionality of the patient's CMC joint 16 and actions which involve the thumb e.g. gripping objects, compared to an untreated CMC joint 16 or a CMC joint 16 treated using the prior art. These advantages may be provided by the splint 100 being configured to hold the thumb 12 in a desired position, as should become clearer from the following description.

The splint 100 is configured to hold the patient's thumb in a desired position in which the symptoms of arthritis in the CMC joint 16 are at least partially, or completely, reduced. For instance, the first metacarpal 17 is moved into an orientation so that the CMC joint 16 is not in flexion, and the splint 100 provides resistance to the thumb 12 collapsing towards or into flexion.

It should be understood that holding the first metacarpal 17 in the desired orientation does not substantially or completely immobilise the thumb 12 at the CMC joint 16. Instead, the splint 100 may allow some movement of the first metacarpal 17 at the CMC joint 16 e.g. into or towards one or more of extension, adduction and abduction. In addition, the splint 100 may not restrict or hinder movement at the joint between the proximal phalanx and distal phalanx 22 of the thumb 12.

Alternatively, or in addition, the splint 100 may allow movement at the CMC joint 16 towards or into flexion. For instance, this may be enabled by the structure of the splint 100 having a degree of resiliency that can be overcome by the patient's muscles which allow the thumb to push against the cradle and cause the bridge to flex.

The splint 100 includes, or is formed from, a body portion 102.

The body portion 102 may be integrally formed as a single piece, e.g. by moulding or three-dimensional (3D printing). Alternatively, the body portion 102 may be formed from multiple sub-parts which can be connected together to form the splint 100.

In some forms, the body portion 102 may be formed from a thermoplastic material or resin, or other suitable material as would be known to one skilled in the art.

The body portion 102 is shaped to define a cradle, indicated generally as 104, and an anchor, indicated generally as 106.

The body portion 102 is optionally resilient and substantially inflexible, yet has a relatively limited amount of flex in one or more regions. The resiliency and limited flexibility allow the body portion 102 to flex slightly in use, which may facilitate benefits such as an improved range of motion and usability for a patient, fitting of the splint 100 to patients and allowing one size of splint 100 to fit a range of hand sizes.

The cradle 104 includes a first wing 108 and a second wing 110. In use, the first wing 108 and the second wing 110 can each be positioned relative to a surface of the thumb 12 e.g. the inner surface 15. In addition, or the alternative, the cradle 104 can apply pressure to the thumb 12 to facilitate it being pushed towards the desired position and/or to act as a barrier to prevent the thumb 12 moving towards the palm 8 e.g. towards or into flexion.

The first wing 108 and the second wing 110 are configured to at least partially wrap around the thumb 12. The ends 108A and 110A of the first wing 108 and the second wing 110 respectively are distal to the body portion 102 and are spaced apart from each other and define a gap (unnumbered).

It is also envisaged that the cradle 104 may be in the form of a continuous loop that surrounds the thumb 12 or a sheath which fits over top of the thumb 12.

The anchor 106, which is optionally provided on the body portion 102. The anchor 106 is configured to be positioned, in use, adjacent to and press against a surface of the patient's body that is on the proximal side of the CMC joint 16.

It can also be seen that the anchor 106 is configured to press against an outer surface 19 of the thumb 12 on at least the proximal side of the CMC joint 16.

Providing an anchor 106 on the proximal side of the thumb 12 may facilitate the benefits of the present technology. For instance, it provides a relatively or completely fixed point from which leverage may be applied to the thumb 12 to hold it in or towards the desired orientation e.g. for the splint 100 one or more of extension, adduction and abduction. In addition, in the illustrated embodiment, the anchor 106 is structured and/or arranged to be relatively rigid for instance by being formed integrally with the body portion 102 which extends continuously across the portion of the hand with which the anchor 106 is intended to be in contact.

Many prior art splints e.g. for a CMC joint 16 do not anchor on the proximal side of the CMC joint 16. Instead, the prior art attempts to anchor from the hand itself e.g. by wrapping around the palm 8 and back 10; this however requires a bulkier and more obtrusive splint than that provided by the present technology. Furthermore, the prior art may be more limited in the orientation to which it can place and hold the thumb 12, thereby providing less advantageous treatment of arthritis than what the present technology may provide. Some prior art splints may try to anchor in the region of the hand around the thumb 12. However, these do not have a rigid anchor 102 and therefore may be less effective in creating a traction force and/or positioning the joint into a desired orientation.

In addition, the cradle 104 has a distal edge 104A which is configured to be placed adjacent to and bear against a surface of the patient's body which is distal to the joint e.g. on the distal side of the axis of rotation (not indicated in the Figures) of the CMC joint 16. In the illustrated embodiment, the cradle 104 bears against, or distal to, a joint which is distal to the CMC joint e.g. the joint between the metacarpal 17 and proximal phalange 21. This may assist in creating a lever arm so that attempted flexion of the CMC joint 16 creates a traction force that is applied to the CMC joint 16. For instance, the traction force may be caused by contraction of the muscles which cause flexion of the joint between the metacarpal 17 and the proximal phalange 21.

The traction force may lift the metacarpal 17 in the CMC joint 16. The inventor has surprisingly found that the application of a lever arm using the present technology may provide a number of benefits such as improving a patient's grip strength or reducing pain while trying to use/move the CMC joint 16.

As can be seen from the Figures, the body portion 102 defines an aperture 118, which allows the body portion 102 to be slipped over the thumb 12. For instance, as illustrated the body portion 102 is shaped to define a first arm 114, and optionally a second arm 116, which extend from the anchor 112.

The first arm 114 and the second arm 116 are each shaped or configured to confirm to the surfaces of the patient's hand in regions surrounding the CMC joint 16. For instance, the first arm 114 and second arm 116 are generally curved through multiple planes to conform to the surfaces of the hand as required e.g. the volar and dorsal surfaces respectively around the CMC joint 16. The shape of the first arm 114 and second arm 116 allows them to, in use, each conform to a respective side of the thumb 12 and together define the aperture 118.

It is also envisaged that the body portion 102 may have only one arm, or that the first arm 114 and the second arm 116 may not meet e.g. one arm only partially wraps around the volar and dorsal surfaces of the hand around the CMC joint 16 in use.

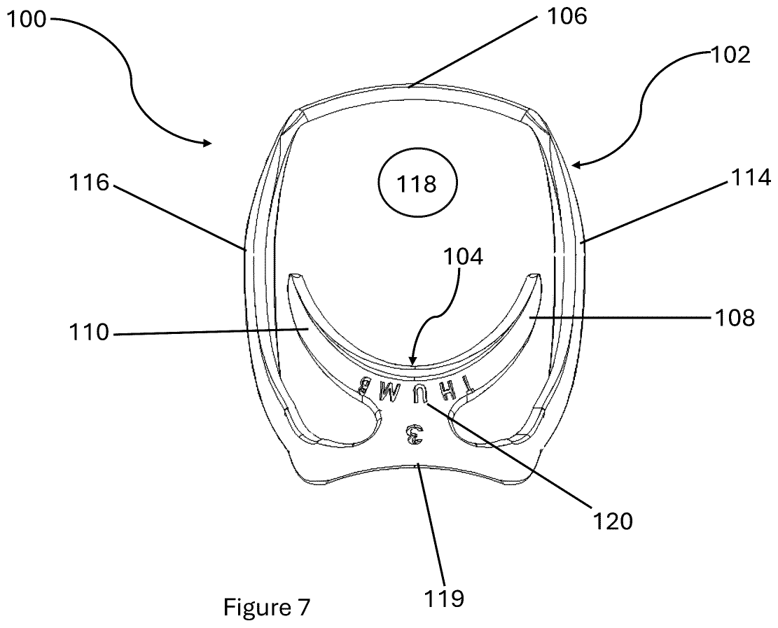

As is perhaps best seen in FIG. 7, the first arm 114 and the second arm 116 connect to each other on a distal side of the aperture 118.

The body portion 102 is structured to provide a second anchor 119 which is optionally located on the distal side of the aperture 118 from the anchor 112. The second anchor 119 may be shaped and configured to bear against a surface of the patient's body which is distal to the joint being treated. For instance, in the embodiment of the splint 100 this may be in the area of one or more of the metacarpal (unnumbered) and proximal phalanx (unnumbered) of the index finger 14.

The cradle 104 extends away from the body portion 102, from an area around the second anchor 119. For instance, in the illustrated embodiment, the cradle 104 is connected to the body portion 102 by a bridge 120. The bridge 120 provides a space between the cradle 104 and the arms 114, 116, and second anchor 119 can assist with positioning the cradle 104 with respect to the thumb 12.

In addition, at least one of the cradle 104 and the bridge 120 may be able to flex as it/they has/have a degree of resiliency which may facilitate the position of the cradle 104 to be adjusted e.g. during fitting. Alternatively, or in addition, the bridge 120 may be able to flex along its length and/or at a single point e.g. a discrete hinge is provided or formed in the bridge 120 or where it meets the arms 114, 116 and/or the second anchor 119.

The flex may be in response to movement of the patient's thumb thereby improving functional movement for the patient while still alleviating or eliminating the symptoms of arthritis in the thumb by holding the thumb 12 in the desired position e.g. in or towards one or more of extension, abduction and adduction.

However, the cradle 104 could extend away from the arms 114 and 116 closer to the anchor 112. It is also envisaged that a bridge 120 may not be provided and that the cradle 104 could be formed into one or more of the first arm 114 and the second arm 116 without a distinct structure to achieve a desired position for the cradle 104.

In some forms, the splint 100 may include a liner indicated generally as 122.

The liner 122 is optionally formed from a material which is one or more of soft, flexible, breathable, and water resistant. For instance, the liner 122 may be a foam material such as an open cell PVC foam.

The liner 122 may be permanently attached to the body portion 102 e.g. by an adhesive, or removably attached e.g. by a semi-permanent adhesive or a fastener such as a loop and hook fastener sold under the trade name Velcro™.

The liner 122 may also be provided in two or more parts, for instance a second liner portion 123 can be provided. The liner 122 and the second liner portion 123 can be attached to the body portion 102 separately and/or spaced apart from each other around the body portion 102. Therefore, the liner 122 and second liner portion 123 can together provide cushioning at spaced apart portions around the body portion 102 - thereby potentially improving patient comfort and compliance.

In addition, or the alternative, the liner 122 and the second liner portion 123 may be different thicknesses, or made of different materials to each other. This may facilitate benefits such as size adjustment, adaptability or sizing to fit patient needs.

The splint 100 includes a fastener 124 configured to facilitate holding the splint 100 in position on a patient's hand. In the illustrated embodiments, the fastener 124 is provided in the form of a strap, which attaches into a connection portion e.g. a slot 126 formed in the body portion 102. In this embodiment, the strap 124 can wrap around the patient's arm 6 in the region near the wrist 4.

However, other forms of fastener are envisaged. For instance, the fastener may be provided by an adhesive applied to the liner 122 or body portion 102, and which can adhere to the patient's skin. Yet further embodiments envisaged by the present technology include Velcro™, glue or a suction-cup type fitting.

The fastener 124 is configured to hold the splint 100 in position over the patient's thumb, and may reduce or eliminate it slipping off the thumb 12 and/or rotating around the thumb 12.

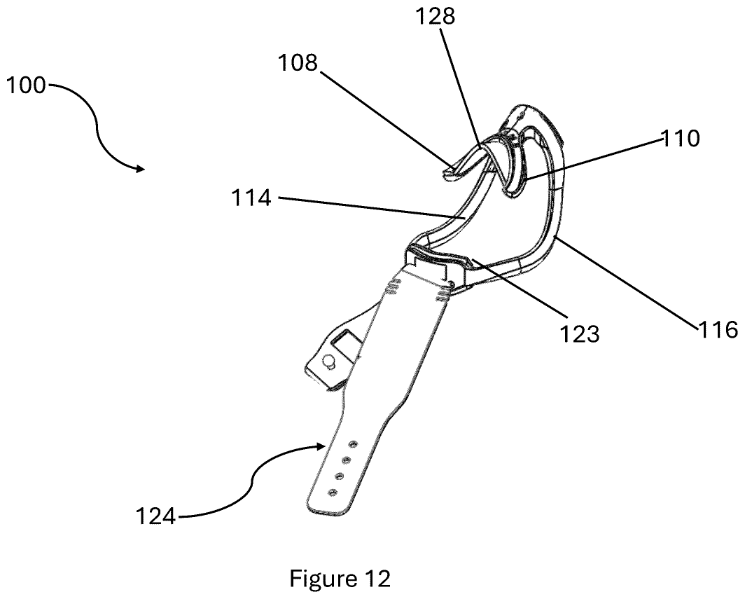

Referring now to FIGS. 12 to 15 which illustrate additional features of the splint 100. The liner 122 is shaped and/or configured to extend beyond the edges of the wings 114, 116 that define the cradle 104, to provide an overhang (128).

The overhang 128 provides an extended surface area for the splint 100 beyond the more resilient and inflexible structure provided by the body portion 102.

The overhang 128 may allow the splint 100 to contact the thumb 12 in a relatively larger area than in an embodiment without the overhang 128. In addition, it may be more flexible, facilitating the thumb 12 to move from a position in which it is held by the splint 100 towards abduction.

The overhang may also facilitate the splint 100 better fitting a wider range of patient's hand sizes. For instance, it may provide a combination of contact and resistance to the thumb moving towards abduction for a wider range of hand and thumb sizes.

Additional structural features of the splint 100, its component parts, their interactions and functions, should become clearer from the following description.

### Splint Fitted to a Patient's Hand

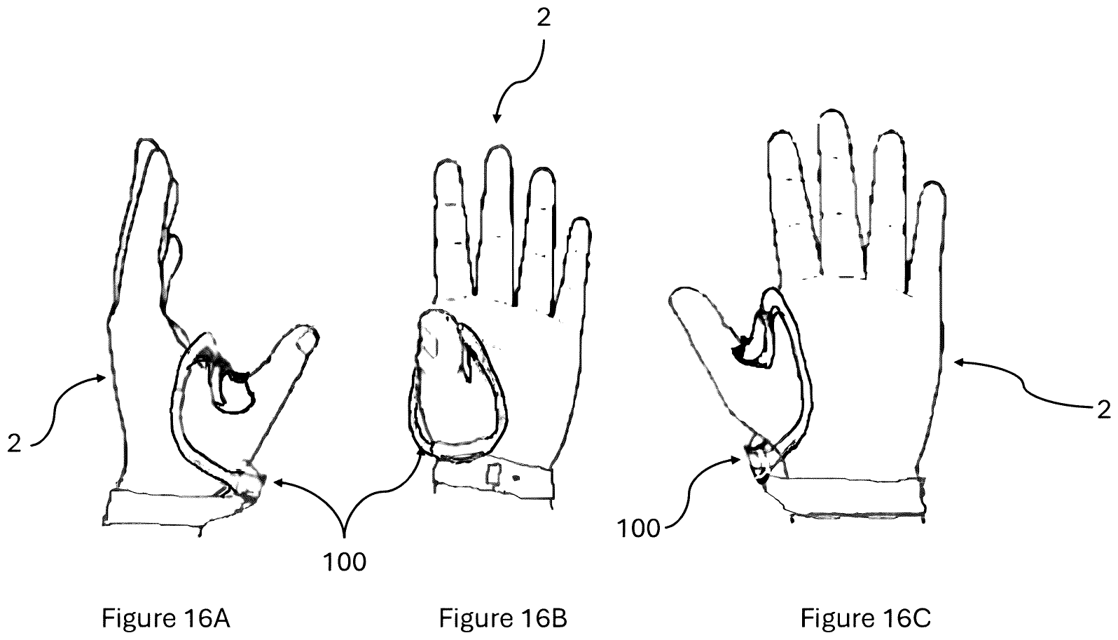

Referring now to FIGS. 16A to 16B which show views of splint 100 according to the present technology fitted to a patient's hand 2, with the thumb 12 in different positions e.g. abduction (FIG. 16A), adduction (FIG. 16B), and extension (FIG. 16C).

As can be seen in FIG. 16A, the splint 100 when fitted to patient's hand will assist to hold the thumb 12 in or towards abduction. In this position, the inner surface 15 of the thumb 12 is in contact with the splint e.g. one or more of the cradle 104 and the overhang 128. The splint 100 applies resistance to the thumb 12 moving inwards across the palm 8 e.g. into or towards flexion.

However, the patient is still able to move the thumb 12 into or towards adduction (as is illustrated in FIG. 16B).

In addition, or in the alternative, the open nature of the cradle 104 may allow a patient to move their thumb into or towards extension as is illustrated in FIG. 16C. For instance, there is a gap, indicated by line 130 in FIG. 16C which indicates how the thumb has moved away from the cradle.

It should be appreciated that the shape and or configuration of the splint 100 allows it to provide support to the thumb to hold it in or towards adduction while preventing or limiting movement towards flexion. However, the splint 100 still allows some desired movements as may be achievable based on the status of disease in the CMC joint 16.

In addition, the splint 100 is structured to allow movement at the first metacarpo-phalangeal joint of the thumb. As a result, a patient may still have an improved grip compared to when the splint 100 is not worn, or prior art splints.

As discussed above, the splint 100 contacts the thumb 12 distal to the CMC joint 16 e.g. in an area distal to the CMC joint 16, and optionally in the area of or distal to the first metacarpo-phalangeal joint of the thumb 12. The cradle 104 therefore provides a lever arm, such that contraction of muscles to move the first metacarpo-phalangeal joint towards flexion may may lift the proximal end of the metacarpal in the CMC joint 16. As result, this may relieve the joint, thereby reducing pain and/or facilitating a relatively stronger grip than is possible without the splint 100.

It should be appreciated that lifting the proximal end of the metacarpal in the joint is a distinct movement to positioning the CMC joint in a desired orientation. It is achieved by the structure of the splint 100 to create a lever arm due to where the splint contacts the patient. This may also be supplemented by the structure and function of the anchor and/or the bridge 120.

The resiliency of the splint 100 e.g. in the cradle 104 and/or the bridge 120 may also facilitate pain reduction and relatively improved grip strength for a patient. For instance, the feature may allow the metacarpal of the thumb to be moved towards flexion (even to a small degree) while minimising movement at the CMC joint 16.

### Method of Fitting a Splint

It should be appreciated from the foregoing description that the present technology encompasses a method of treating arthritis in a patient's thumb using a splint as substantially described herein. The method may include one or more of the following steps in any order:
(a) Selecting an appropriate size splint for the patient's thumb;
(b) Positioning the Splint Relative to the Thumb;
(c) Moving the splint and/thumb relative to each other so as to insert the patient's thumb through the aperture. This can continue until the anchor is positioned relative to, and on a proximal side of the CMC joint 16 and the second anchor 119 is positioned relative to the metacarpus and proximal phalanx (not numbered in the Figures) of the index finger 14.
(d) Applying sufficient pressure to the splint to cause move the patient's thumb towards into a desired position e.g. into or towards abduction; and
(e) Attaching the splint 100 to the hand using the fastener.

Once fitted to the patient's hand, the splint provides resistance to the thumb moving towards flexion. However, the cradle 104 is shaped to allow movement of the thumb 12 at the joint between the proximal phalanx 17 and distal phalanx (not numbered in the Figures). Therefore, the patient is still able to grip objects while wearing the splint 100. Furthermore, the ability of the splint 100 to hold the thumb 12 in or towards abduction, e.g. as illustrated in FIG. 16A, may improve grip strength and/or reduce and eliminate patient pain and discomfort e.g. due to arthritis of the CMC joint 16.

Furthermore, the cradle applies pressure to the thumb at a location distal to the first metacarpal-phalangeal joint while not inhibiting or preventing flex and the joint between the proximal and distal phalanxes of the thumb. This may provide a lever arm to lift the proximal end of the metacarpal in the CMC joint 16 to reduce pain and/or provide a relatively improved functional grip for the patient. That lever arm may be present due to the structure of the splint 100, and/or created on contraction of the muscles in the thumb to flex the metacarpal and/or proximal phalange.

### Second Embodiment of a Slint

Referring now to FIG. 17A which illustrate a boxer's fracture splint 200 configured for treating a boxer's fracture in a patient's hand 2, and FIG. 17B which shows anatomy of the hand 2 to facilitate understanding of the technology.

In FIG. 16, the boxer's splint 200 is fitted to a patient's hand 2.

A boxer's fracture is a break in the fifth metacarpal 204 of the hand 202, and forms a first bone fragment and a second bone fragment (neither numbered in the Figures), which a misaligned. To facilitate optimum healing of the boxer's fracture it is desirable to reduce the misalignment and hold the bone fragments in a desired orientation relative to each other.

The boxer's fracture splint 200 may take any suitable form. However, in the illustrated embodiment, the boxer's fracture splint 200 has a body portion 202 which is configured as a clamp which fits to the lateral edge 206 of the hand 2.

The boxer's fracture splint 200 has an anchor 208 which is configured to contact the palm surface of the hand 2 at a location which is proximal to the fracture site (not indicated in the Figures). In addition, the anchor 208 contacts the hand 2 proximal to the facture site e.g. adjacent to the fifth metacarpal 204.

The boxer's fracture splint 200 includes a bearing portion (indicated generally as 211), which is configured to apply pressure to an area of the hand which enables it to position the second bone fragment to reduce the fracture of the fifth metacarpal 204.

In addition, the boxer's fracture splint 200 includes a cradle (indicated as 212 in the Figures). The cradle 212 is configured to contact the patient's hand in an area which is distal to the fifth metacarpo-phalangeal joint 215, and optionally proximal to the fifth interphalangeal joint 214.

The cradle 212 is configured to ensure that it does not hinder or prevent flex in the joint between the proximal and distal phalange of the little finger.

The structure of the splint 200 creates a lever arm to lift the fifth metacarpal 210 in the fifth metacarpal joint 210 (the CMC joint of the little finger). Therefore, the splint for a fracture 200 may allow or facilitate relatively normal movement of the little finger while treating a boxer fracture.

### Third Embodiment of a Splint

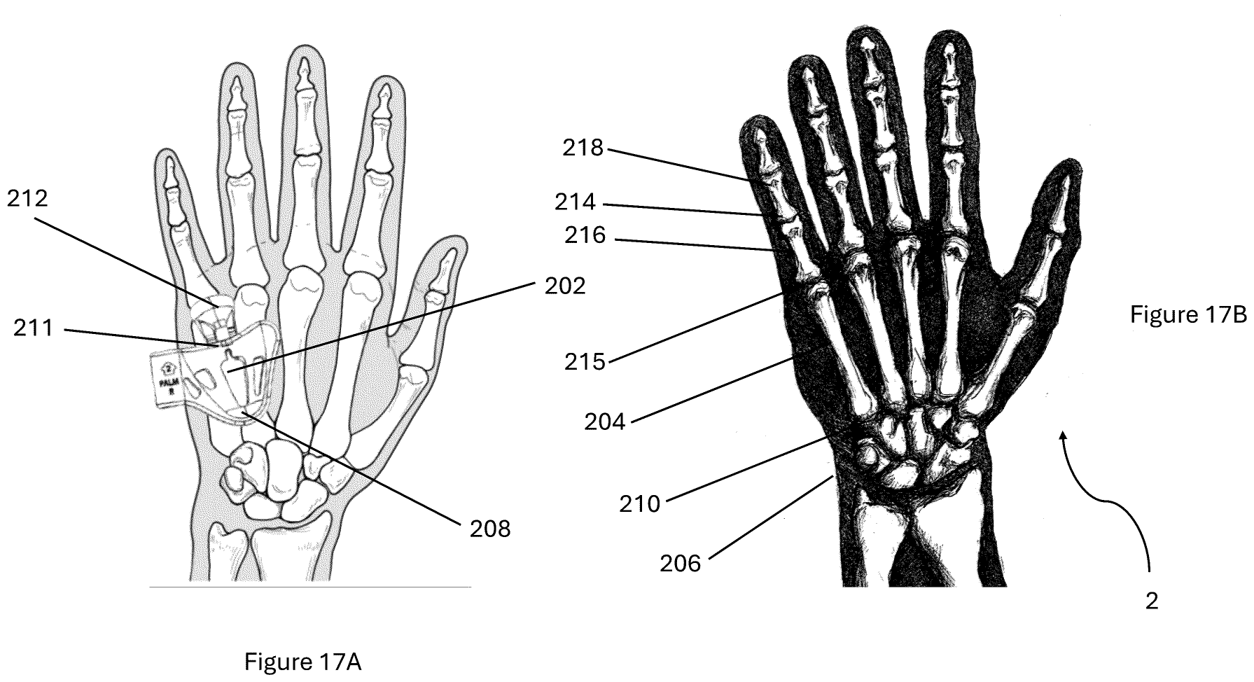

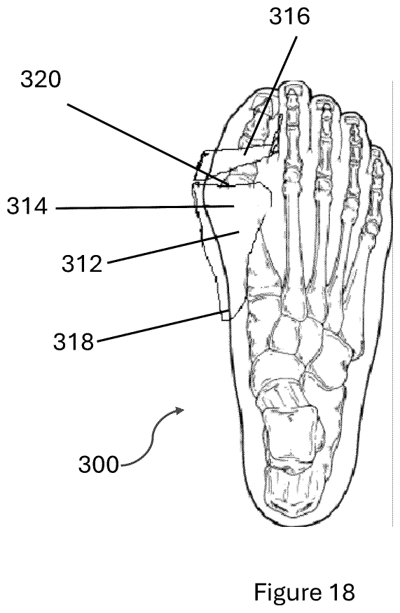

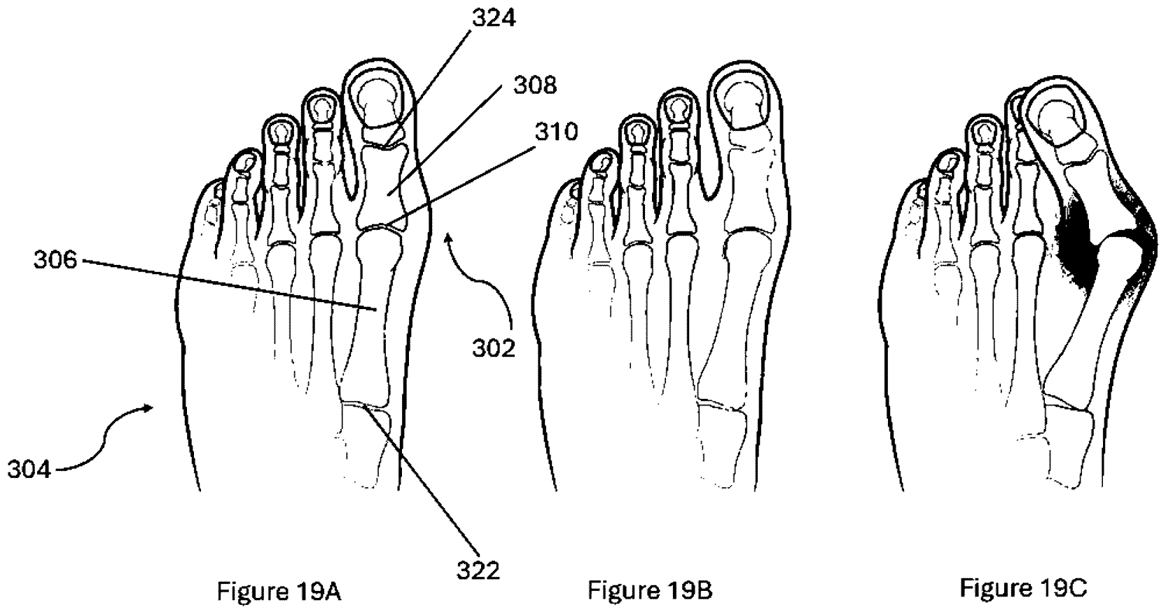

Referring now to FIG. 18 which shows an embodiment of a splint according to an aspect of the present technology, in the form of a hallux valgus splint 300 configured for treating a hallux valgus deformity, and FIGS. 19A-C which illustrate anatomy of the foot to assist with understanding of the technology.

The hallux 302 is the medial digit of an animal's lower limb, and in humans refers to the big toe of the foot 304.

A hallux valgus deformity, often referred to as a bunion, is a deformity of the base joint of the hallux (big toe), between the first metatarsal 306 of the foot 304 and the first proximal phalanx 308 of the foot 304.

The hallux valgus splint 300 is configured to be positioned adjacent the patient's hallux and apply pressure to the surfaces of the foot 304 surrounding the first metatarsal-phalangeal joint 310 to thereby position the first proximal phalanx 308 in a desired orientation with respect to the first metatarsal 306. In doing so, the hallux valgus splint 300 can hold the first metatarsal-phalangeal joint 310 in a desired orientation e.g. in or towards a relatively normal position or hallux varus.

The hallux valgus splint 300 has a body portion 312 which is shaped and configured to define an anchor 314 and a cradle 316.

The anchor 314 is configured to be positioned adjacent to and bear against a surface of the foot 304 which is proximal to the first metatarsal-phalangeal joint 310.

As can be seen in FIG. 18, the body portion 312 is shaped to have a proximal end 318 and a distal end 320. The anchor 314 is optionally formed into the distal end 320, and therefore the hallux valgus splint 300 may be relatively short.

However, it is also envisaged that the hallux valgus splint 300 may have a relatively elongate shape such as that illustrated. That can facilitate a relatively longer contact surface with the foot 304 e.g. extending along the foot 304 to contact adjacent to the metatarsal-medial cuneiform joint 322. This relatively longer contact surface, and the portions of the foot which it contacts, may enable the splint 300 to facilitate more effective realignment of the first metatarsal-phalangeal joint 310.

The cradle 316 is configured to be positioned adjacent to and bear against a surface of the first proximal phalanx 308. While not shown in FIG. 17, the cradle can at least partially wrap around the first proximal phalanx 308, contacting one or more of the superior and inferior surfaces. For instance, the cradle 316 may define a sleeve or loop having an aperture into which the digit of the hallux can be inserted.

The hallux valgus splint 300 may also include a fastener (not illustrated in the Figures. The fastener may be a strap which can loop around the patient's foot and connect to the splint e.g. at or towards the proximal end of the body portion. Alternatively, the fastener may be an adhesive which stick the splint to the patients'foot.

The body portion 312 is structured to be relatively resilient. Accordingly, the hallux valgus splint 300 can provide sufficient force into the bones forming the metatarsal-phalangeal joint to hold them in a desired orientation, but can flex to allow at least some movement at the first metatarsal-phalangeal joint 310 during actions such as walking or generally flexion.

The hallux valgus splint 300 is configured to apply force to position the bones of the first metatarsal-phalangeal joint 310 in or towards a relatively normal state as indicated in FIG. 19A or a hallux varus. For instance, this may be used to treat a hallux valgus deformity such as that shown in FIG. 19B or 19C.

The hallux valgus splint 300 may also be configured to create a traction force on flexion of muscles surround the first metatarsal-phalangeal foot. For instance, the cradle 316 may be shaped and configured to define a distal surface (not indicated in the Figures) which is positioned relative to and contacts a surface of the hallux which is distal to the first inter-phalangeal joint 324 e.g. an inferior surface of the digit that is distal to the axis of rotation of the inter-phalangeal joint 324. The traction force can assist to lift the first proximal phalanx 310 in the first metatarsal-phalangeal joint 308, thereby a patient's reducing pain and discomfort while also facilitating movement.

It will be appreciated by persons skilled in the art that numerous variations and/or modifications may be made to the above-described embodiments, without departing from the broad general scope of the present disclosure. In addition, features or the described embodiments of the present technology may be combined to form yet further embodiments of the technology as are envisaged by the scope of the present disclosure.

The presently described embodiments are, therefore, to be considered in all respects as illustrative and not restrictive.

Therefore, the following is claimed:

1. A splint to treat a patient's joint (“the treated joint”), wherein the treated joint has an axis of rotation, wherein
the splint includes a cradle and an anchor, and wherein
the splint is, in use, positioned with respect to the treated joint so that:
the cradle is adjacent to and bears against a surface of a patient's body that is distal to the axis of rotation, and
the anchor is adjacent to and bears against a portion of the patient's body that is proximal to the axis of rotation; and
the splint is configured to position bones forming the treated joint in a desired orientation and at least partially restrict movement at the bones away from the desired orientation; and further wherein
the cradle and the anchor together define a lever arm based on the points of the patient's body with which the anchor and cradle bear which provides a traction force to the treated joint on contraction of muscles in the patient's body.

2. The splint of claim 1, wherein the splint further comprises a fastener configured to attach the splint to the patient's body relative to the treated joint.

3. The splint of claim 2, wherein the splint includes a liner.

4. The splint of claim 3, wherein the liner is formed from a material which is at least one of soft, flexible, water resistant, and breathable.

5. The splint of claim 1, wherein treated joint is the patient's CMC joint, and further wherein the cradle is configured to be positioned and bear against a surface of the patient's thumb, and wherein the anchor is configured to bear against a surface of the patient's body which is proximal to the CMC joint.

6. The splint of claim 5, wherein the cradle is configured to bear against a surface of the patient's thumb on the distal side of the CMC joint.

7. The splint of claim 6, wherein the cradle is configured to bear against a surface of the patient's thumb that is at least one of substantially adjacent to or distal to, a joint between the metacarpal and the proximal phalange of the patient's thumb, and further wherein contraction of muscles in the patient's thumb to cause flexion in the joint between the metacarpal and the proximal phalange causes the traction force.

8. The splint of claim 7, wherein the cradle is defined by a first wing and a second wing, and further wherein the first wing and the second wing are configured to at least partially wrap around the patient's thumb.

9. The splint of claim 8, wherein the cradle is structured to allow at least partial movement of the bones from the desired orientation further towards one or more of extension, and abduction.

10. The splint of claim 9, wherein the splint includes a first arm and a second arm, wherein the first arm has a curved shape which allows it to confirm to the inner surface of the patient's hand in the region adjacent to the CMC joint, and further wherein the second arm has a curved shape which allows it to conform to the outer surface of the patient's hand in a region adjacent the CMC joint.

11. The splint of claim 10, wherein the splint includes a bridge which connects the cradle to one or more of the first arm, the second arm, and the bearing portion.

12. The splint of claim 11, wherein the bridge is at least partially resilient and able to flex in use.

13. The splint of claim 12, wherein the amount of flex in the bridge is relatively minimal.

14. The splint of claim 13, wherein the cradle includes a sling.

15. The splint of claim 14, wherein the sling is a region of the cradle which has a relatively higher degree of flexibility compared to other regions of the cradle.

16. The splint of claim 15, wherein the sling is provided by a layer of material attached to the cradle.

17. The splint of claim 16, wherein the layer of material extends beyond an edge of the cradle and is unsupported on an inner surface and an outer surface and along a side edge of the sling.
